(19) United States
(12) Patent Application Publication
(10) Pub. No.: US 2006/0101539 A1
(43) Pub. Date: May 11, 2006

(54) Cryopreservation of cells

(76) Inventors: William Michael Ainley, Carmel, IN (US); Cory Michelle Leatherman Larsen, Zionsville, IN (US); Min Lu, Carmel, IN (US); Liu Yin Shen, Westfield, IN (US); Pon Samuel Jayakumar, Carmel, IN (US); Robbi Janette Garrison, Fillmore, IN (US); Dayakar Reddy Pareddy, Carmel, IN (US); Jeffrey Richard Beringer, Carmel, IN (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/266,880

(22) Filed: Nov. 4, 2005

Related U.S. Application Data
(60) Provisional application No. 60/625,401, filed on Nov. 5, 2004.

(51) Int. Cl. C12N 5/04; C12N 15/82; A01H 1/00

(52) U.S. Cl. 800/278; 435/419

(57) ABSTRACT

The present invention relates to methods for the cryopreservation of transformed and non-transformed cells. Also provided by the subject invention are methods of recovering cells that have been cryopreserved. Cultures of cells that have been successfully recovered from cryopreservation are also provided.

### CROSS-REFERENCE TO RELATED APPLICATION

This application claims the benefit of U.S. Provisional Application Ser. No. 60/625,401, filed Nov. 5, 2004.

### FIELD OF INVENTION

The present invention relates to methods for the cryopreservation of transformed and non-transformed cells. Also provided by the subject invention are methods of recovering cells that have been cryopreserved. Cultures of cells that have been successfully recovered from cryopreservation are also provided.

### BACKGROUND OF THE INVENTION

“Master Seed” principles for biopharmaceutical and bioagrochemical production utilize live organisms as part of the manufacturing procedure and rely on some basic tenets: 1) a single culture of defined origin and passage history is preserved with defined characteristics of cell phenotype and desired manufacturing features; 2) preservation, typically cryopreservation, is long lasting (spanning several years or more); 3) the cell can be recovered, expanded, passaged indefinitely into “working seed” and subjected to another period of cryopreservation (a principle that requires robustness of the cell; and 4) the cell does not lose the defined characteristics of cell phenotype and desired manufacturing features found prior to the initial cryo-state after a defined number of passages.

The art related to cryopreservation of plant cells is devoid of teachings related to those features needed for use of a biological agent in a biopharmaceutical manufacturing environment. Specifically, any technique devised for prolonged storage of viable biological agents should preferably meet the criteria of: 1) the storage method must provide biological agents that are stable over long periods of times (years); 2) the storage conditions should not alter the biological agent needed for the manufacturing process; and 3) the agent should be readily available for regrowth once removed from storage and expandable into working seed that can be regrown.

Little information is available in the prior art related to lengths of cryopreservation (often measured in months or even hours) and there is limited information on the whether cells can be grown indefinitely or at least to a desired number of passages under normal culture conditions. Additionally, very little data reveals genetic and product stability of target gene(s) or gene product(s) over a prolonged storage or prolonged cultivation after removal from storage, both from a primary Master Seed Stock and an expanded and re-cryopreserved Working Seed Stock.

Thus, the art is in need of methods or sets of treatments for long term storage of plant cells that provide for the long term growth, re-cryopreservation, and stability of biomanufacturing target components under master seed principles.

### SUMMARY OF THE INVENTION

The present invention relates to methods for the cryopreservation of transformed and non-transformed cells. Also provided by the subject invention are methods of recovering cells that have been cryopreserved. Cultures of cells that have been successfully recovered from cryopreservation are also provided.

### BRIEF DESCRIPTION OF THE FIGURES

FIGS. 1A-1B. Effect of the frequency of cell transfer on the Percent Recovery (FIG. 1B) and Percent Healthy Callus (yellow callus; FIG. 1A).

### DETAILED DESCRIPTION OF THE INVENTION

The subject invention provides methods for the cryopreservation of transformed or non-transformed cells. In certain embodiments of the subject invention, the methods provide for the formation of cryopreservation compositions and methods for cryopreserving transformed or non-transformed eukaryotic cells.

Thus, one embodiment of this invention provides methods of forming a cryopreservation composition comprising transformed (or non-transformed) eukaryotic cells. These methods comprise the steps of:
a) growing transformed (or non-transformed) cells on/in selectable media;
b) inoculating a culture flask containing culture medium with said cells to form a liquid culture and passaging said liquid culture of transformed (or non-transformed) cells at least 1, 2, 3, 4, 5, 6, 7, 8, 9, or 10 times;
c) recovering said passaged cells; and
d) adding said recovered cells to a cryopreservation media to form a cryopreservation composition.

Another embodiment of the subject invention provides methods for the cryopreservation of transformed (or non-transformed) eukaryotic cells comprising the steps:
a) growing transformed (or non-transformed) cells on/in selectable media;
b) inoculating a culture flask containing culture medium with said cells to form a liquid culture and passaging said liquid culture of transformed (or non-transformed) cells at least 1, 2, 3, 4, 5, 6, 7, 8, 9, or 10 times;
c) recovering said passaged cells;
d) adding said recovered, transformed (or non-transformed) cells to a cryopreservation media to form a cryopreservation composition; and
e) cryopreserving said cryopreservation composition.

Yet another embodiment provides a method for cryopreserving transformed (or non-transformed) cells comprising:
a) growing transformed (or non-transformed) cells on/in selectable media for 1-10 days;
b) inoculating a culture flask containing culture medium with said cells to form a liquid culture;
c) culturing said liquid culture to about mid-log growth phase;
d) withdrawing a first volume (VOL1) of said liquid culture and inoculating it into culture flask containing second volume (VOL2) of culture medium;
e) repeating step d) at least 1, 2, 3, 4, 5, 6, 7, 8, 9, or 10 additional times (passaging said cells);
f) recovering said passaged cells;
g) suspending said passaged cells in one volume of a second medium;
h) adding cryopreservation media to the suspended cells provided in step g) to form a cryopreservation composition;
i) cooling said cryopreservation composition; and
j) freezing said cryopreservation composition.

According to the subject invention, one embodiment provides for the cryopreservation of plant cells. Cells derived from monocots or dicots can be cryopreserved according to the subject invention. Thus, transformed and non-transformed monocot or dicot cells can be cryopreserved using the various methods taught herein. In various embodiments of the invention taught herein, transgenic and non-transgenic tobacco and rice cells are cryopreserved, stored and recovered to establish growing cell cultures that retain the genotype and phenotype of the original culture.

Thus, the subject invention provides methods for the cryopreservation of transformed plant cells, optionally under master seed principles. In certain embodiments of the subject invention, the methods are applied to methods for cryopreservation of Nicotina tabacum (NT-1 and BY-2) cells and T309 rice cells under master seed principles. See Biotechnology in Agriculture and Forestry, Eds. T. Nagata, S. Hasezawa, and D. Inze; Springer-Verlag; Heidelberg, Germany; 2004.

The T309 rice cell line was prepared from commercially available rice T309 variety using standard plant tissue culture techniques. Additional transformed and untransformed plant cells that are suitable for the practice of the subject invention are provided in Table 1.

Another embodiment of this invention provides methods of forming a cryopreservation composition comprising transformed plant cells. These methods comprise the steps of:
a) growing transformed plant cells on callus on selectable media;
b) inoculating a culture flask containing culture medium with said plant cells from said callus to form a liquid culture and passaging said liquid culture of transformed plant cells at least 1, 2, 3, 4, 5, 6, 7, 8, 9, or 10 times;
c) recovering said passaged plant cells; and
d) adding said recovered, transformed plant cells to a cryopreservation media to form a cryopreservation composition.

Another embodiment of the subject invention provides methods for the cryopreservation of transformed plant cells. These methods comprise:
a) growing transformed plant cells on callus on selectable media;
b) inoculating a culture flask containing culture medium with said plant cells from said callus to form a liquid culture and passaging said liquid culture of transformed plant cells at least 1, 2, 3, 4, 5, 6, 7, 8, 9, or 10 times;
c) recovering said passaged plant cells;
d) adding said recovered, transformed plant cells to a cryopreservation media to form a cryopreservation composition; and
e) cryopreserving said cryopreservation composition.

Yet another method for cryopreserving transformed plant cells comprises:
a) growing transformed plant cells on callus on selectable media for 1-10 days;
b) inoculating a culture flask containing culture medium with said plant cells from said callus to form a liquid culture;
c) culturing said liquid culture to about mid-log growth phase;
d) withdrawing a first volume (VOL1) of said liquid culture grown to about mid-log phase, inoculating said first volume into culture flask containing a second volume (VOL2) of culture medium to form a passage culture and culturing said passage culture to about mid-log growth phase;
e) optionally, repeating step d) at least 1, 2, 3, 4, 5, 6, 7, 8, 9, or 10 additional times;
f) recovering plant cells from said passage culture;
g) suspending said plant cells in a volume of a second medium (CULT);
h) adding a volume of cryopreservation media (CRYO) to the suspended plant cells provided in step g) to form a cryopreservation composition;
i) cooling said cryopreservation composition; and
j) freezing said cryopreservation composition.

The term “passaging” may be used interchangeably with the phrase “short cycle condition(s)”. Passaging or short cycle conditions is/are described as harvesting (withdrawing) cells during mid-exponential (mid-log) growth; diluting or splitting the cells at mid-exponential growth with fresh culture media, and cultivating the diluted (split) cell culture to mid-exponential growth.

For purposes of this invention, the ordinarily skilled artisan will appreciate that the terms “mid-log” and “mid-exponential” do not refer to the precise mid-point of exponential growth but rather refers to a range around the mathematical mid point. Each round of cultivation to mid-exponential growth is considered one cell passage. Cells to be cryopreserved from suspension can be successfully cryopreserved with only 1 short-cycle (passage) or up to as many 20 short cycles. Three to six short cycles are preferred, and 6 short-cycles are most preferred. The inventors have shown that 6 short-cycles (passages) allows for exceptional recovery of cells from a cryopreserved state for recultivation. Additionally, cells can be cyropreserved multiple times after cultivation as long as cells are placed in suspension under short cycle conditions 1-6 times. Volumes of cells (VOL1) that are added to volumes of fresh media (VOL2) in the diluting or splitting step can vary in ratios of VOL1 to VOL2, where VOL1 and VOL2, independently, range from 1 to at least 20; 1 to at least 10; or 1 to at least 5 (inclusive of fractional values between any of these values). In one embodiment, VOL1:VOL2 is 1:3.

It should be further noted that the each of the methods taught infra can comprise additional method steps. For example, it is possible to thaw cryopreserved transformed plant cells, suspend the cryopreserved cells in culture media (or grow the cells on solid selection media to form calluses) and grow them for use in biomanufacturing processes (e.g., culture the cells in growth vessels such as fermentors, stirred tank reactors and the like). Cells derived from the biomanufacturing process can be subjected to the cryopreservation methods of the subject invention and stored according to master seed principles.

Selectable media and culture media suitable for the growth of non-transformed and transformed monocot and dicot cells are known to those skilled in the art and are readily utilizable by these individuals (see, for example, Difco™ & BBL™ Manual, Manual of Microbiological Culture Media).

When cryopreservation compositions are to be formed, various volumes of culture media (CULT) containing resuspended cells can be mixed with various volumes of cryopreservation media (CRYO). These mixtures are mixed in ratios of CULT:CRYO, where CULT ranges from 1 to 100 (inclusive of fractional values thereof) and CRYO ranges from 1 to 100 (including fractional values thereof). In some embodiments, CULT and CRYO range from 1 to 10. In other embodiments, equal volumes of CULT and CRYO are mixed to form a cryopreservation composition.

The subject invention also provides cryopreserved cells or cell lines produced by any of the aforementioned cryopreservation methods.

In various embodiments of the subject invention, the cells that are to be cryopreserved are not “pretreated” or “precultured” with agents, such as stabilizers, that increase cellular viability by removing harmful substances secreted by the cells into the culture medium as is set forth in the teachings of U.S. Pat. No. 5,965,438 or U.S. Pat. No. 6,127,181 (the disclosures of which are hereby incorporated by reference in their entireties, particularly column 6, line 16 through column 7, line 34 of U.S. Pat. No. 5,965,438 and column 6, line 60 through column 9, line 22 or U.S. Pat. No. 6,127,181). As discussed in the '438 patent, stabilizers pretreatment relates to the removal of harmful substances secreted by cells during growth or cell death. Additionally, the subject invention can exclude the use of pretreatment with one or more “osmotic agents”, ethylene inhibitors and/or membrane stabilizers that are added to cells under culture conditions. Particularly, stabilizers, osmotic agents, ethylene inhibitors and/or membrane stabilizers are not added to already prepared culture medium of the subject invention in a pretreatment protocol while cells are being cultured, although substances identified in the '438 or '181 patent may be a component of the medium previously prepared for the culturing of cells according to the subject invention. Thus, stabilizers, osmotic agents, ethylene inhibitors and/or membrane stabilizers are not added to culture medium (or replenished as necessary) as set forth in the '438 or '181 patents during the culture of the cells (see, for example, U.S. Pat. No. 5,965,438 at column 7, lines 7-16 and U.S. Pat. No. 6,127,181 at column 9, lines 36-47).

Accordingly, the following substances are not added to culture medium, or replenished as needed, as the cells are being cultured: stabilizers such as: reduced glutathione, 1,1,3,3-tetramethylurea, 1,1,3,3-tetramethyl-2-thiourea, sodium thiosulfate, silver thiosulfate, betaine, n, n-dimethylformamide, n-(2-mercaptopropionyl) glycine, β-mercaptoethylamine, selenomethionine, thiourea, propylgallate, dimercaptopropanol, ascorbic acid, cysteine, sodium diethyl dithiocarbomate, spermine, spermidine, ferulic acid, sesamol, resorcinol, propylgallate, mdl-71,897, cadaverine, putrescine, 1,3- and 1,2-diaminopropane, deoxyglucose, uric acid, salicylic acid, 3- and 4-amino-1,2,4-triazol, benzoic acid, hydroxylamine and combinations and derivatives of such agents; agents that hinder or substantially prevent ethylene biosynthesis and/or ethylene action such as: rhizobitoxin, methoxylamine HCl, hydroxylamine analogs, α-canaline, DNP (2,4-dinitrophenol), SDS (sodium lauryl sulfate), Triton X-100, Tween 20, spermine, spermidine, ACC analogs, α-aminoisobutyric acid, n-propyl gallate, benzoic acid and derivatives thereof, ferulic acid, salicylic acid and derivatives thereof, salicylic acid, sesamol, cadavarine, hydroquinone, alar, amo-1618, BHA (butylated hydroxyanisol), phenylethylamine, brassinosteroids, p-chloromercuribenzoate, n-ethylmaleimide, iodoacetate, cobalt chloride and other cobalt salts, bipyridyl, amino (oxyacetic) acid, mercuric chloride and other mercury salts, salicyl alcohol, salicin, nickel chloride and other nickel salts, catechol, pffloroglucinol, 1,2-diaminopropane, desferrioxamine, indomethacin, 1,3-diaminopropane, benzylisothiocyanate, 8-hydroxyquinoline sulfate, 8-hydroxyquinoline citrate, 2,5-norbornadiene, n-ethoxycarbonyl-2-ethoxy-1,2-dihydroquinoline, trans-cyclootene, 7-bromo-5-chloro-8-hydroxyquinoline, cis-propenylphosphonic acid, diazocyclopentadiene, methylcyclopropane, 2-methylcyclopropane, carboxylic acid, methylcyclopropane carboxylate, cyclooctadiene, cyclooctodine, (chloromethyl) cyclopropane and/or silver salts such as silver thiosulfate silver nitrate, silver chloride, silver acetate, silver phosphate, citric acid tri-silver salt, silver benzoate, silver sulfate, silver oxide, silver nitrite, silver cyanate, lactic acid silver salt, silver pentafluoropropionate, silver hexafluorophosphate, silver salts of toluenesulfonic acid and combinations thereof; membrane stabilizers such as compounds that intercalate into the lipid bilayer (e.g. sterols, phospholipids, glycolipids, glycoproteins) or divalent cations.

### EXAMPLE 1

### Cryopreservation

Media and components used in this Example are set forth in Tables 2-4. Vendors supplying the components are also indicated. Cells to be cryopreserved are grown in a shaker flask at 25° C. containing NT1 media; the cells are passaged at a 1:3 split (or 30% inoculum) in mid-log (mid-exponential) growth phase (3-4 days after inoculation of culture flasks) for a minimum of 1-10 passages (one embodiment contemplates a minimum of 6 passages). The outside of the flask is cleaned with 1% sodium hyperchlorite solution prior to transfer to a sterile biosafety cabinet, the outside of the flask is then wiped with sterile alcohol pads before transferring the cells to a sterile 225 ml centrifuge tube. Cells are centrifuged at 1000 RPM for 1 minute @ 4° C. and the supernatant is removed with a sterile pipette. The cells are resuspended in the starting volume with appropriate culture media and transferred to a sterile 1 liter Erlenmeyer flask where an equal volume of cryopreservation media is added to the suspension and gently swirled.

The cells are then cryopreserved by gently shaking the cells suspension (130 RPM) in an orbital shaker at 2-7° C. for 1 hour and then transferred on ice to a biosafety cabinet and wiped down with sterile alcohol pads. Cells are immediately dispensed into cryovials using an automatic pipettor under sterile conditions. Each vial receives 2.5 ml of the cell suspension and is immediately placed into the canes to be used for storage in liquid nitrogen; loaded canes should be held at 2-7° C. until time of freezing. The canes are then transferred to a rate control freezer. The freezing process starts with 15 minutes at 4° C., followed by a continual drop in temperature from 4° C. to negative 40° C. at a rate of negative 0.5° C. per minute. The canes are then removed and placed in storage racks precooled on dry ice; as soon as all canes are loaded in storage racks the racks are immediately placed into a liquid nitrogen storage tank using the gas phase.

To recover cells from cryopreservation for use in various bioprocesses, cryovials are removed from liquid nitrogen storage, quickly removed from the storage canes and placed into a 45° C. water bath. The vials are swirled for approximately 2.5 minutes and cells are suspended by inverting the vial. When cells are fully suspended transfer the vials to a biohazard cabinet, wipe of the vial with a sterile alcohol pad and pour the contents onto a stack of 10 sterile Whatman papers in a Petri dish; cover the Petri dish and allow the cryopreservation media to absorb out of the cells for a minimum of 2 minutes. Transfer the top filter to a Petri dish containing NT1 agar media with a lid (make sure there are no bubbles between the Whatman paper and agar), wrap the NT1 agar plate one time with 3M surgical tape and hold the containers at 25° C. Minimal growth of callus should be detectable after 4-5 days. Table 1 shows several different transgenic cell lines for NT-1 cells expressing the hemagglutinin/neuraminidase (HN) protein from Newcastle Disease Virus (NDV), the hemagglutinin (HA) of avian influenza (AIV), heat labile toxin of Escherichia coli and VP2 protein of Infectious Bursa Disease Virus (IBDV). All have been successful regardless of the gene expressed or promoter system utilized. Additionally, data demonstrated that using 3M surgical tape has a significant impact on the growth of NT1 cells as compared to the use of plastic tape or film (e.g., NESCO film).

### EXAMPLE 2

### Thawing of Cryopreserved Tobacco Cells

Cells can be thawed as individual tubes or pools of tubes. Vials are removed from the storage unit and placed on dry ice. They are thawed by immersing them in a 45° C. water bath, gently moving the tube rack in the bath to help facilitate rapid and uniform thawing of the vials. After ˜2.5 minutes (until just thawed), vials are gently inverted 3 times to mix the cells which have settled to the bottom of the tube. In a laminar flow hood, 2 ml of cells from pooled vials, or individual tubes, are pipetted onto stacks of 8-10, sterile 70 mm #4 Whatman filter papers in sterile Petri dishes, covered and allowed to drain for 2 minutes.

TTC viability staining is done at this time on a small amount of cells (˜0.5 ml). After draining for 2 minutes, the top filter with cells is transferred to semisolid NTB 1 media, without bialaphos selection. The media plate is then wrapped with 3M tape and incubated in the dark at 25° C. for 3 days. After 3 days, the 3M tape is replaced with Nesco film. At 7 days, the filter with cells is transferred to a new NT1 media plate and wrapped with Nesco film. Cell growth is evident in approximately 7 days. After an additional 7 days, cells are transferred from the filter and onto new semisolid NT1 plates with bialaphos selection agent. Suspensions are initiated as needed once sufficient cell mass accumulates.

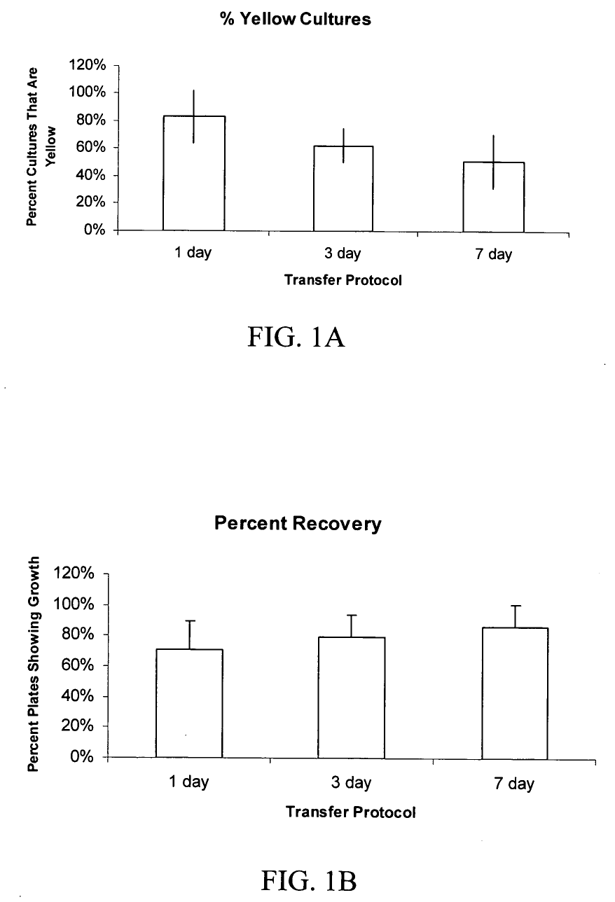

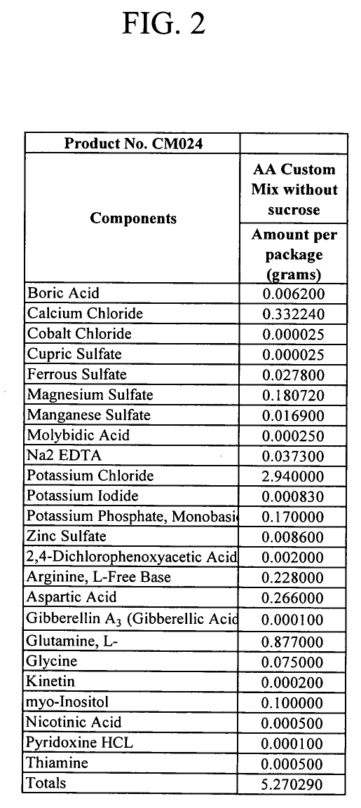

A first set of experiments tested the reproducibility of cell plating after cryopreservation and the best post-thaw treatments. For testing reproducibility, three repetitions of eight tubes were thawed, the cells combined and plated onto eight plates. The other parameter tested was the frequency of transfer of filters containing the cells to fresh plates. Literature references suggested that more frequent transfers can enhance recovery of cells (by removing components in the cryoprotectant); however, there is also a protective effect provided by some components of the cryoprotectant. The three transfer schemes tested included “1 day transfers” (transferred every day for the first three days, transferred after 3 days and then weekly), “3 day transfers” (transferred twice at three day intervals, followed by weekly transfers) and “7 day transfers” (transferred weekly). The results of the experiment were scored based on level of recovery and the color of the cells after two weeks (FIG. 1).

Cells from all treatments testing different times of draining recovered very quickly (5 days). Subjective evaluation of the plates indicated that the cells drained for 1 or 2 min recovered better than the other treatments, although the difference was not large.

Protocol Scale-Up

For testing scale-up, the steps in the procedure were scaled up proportionally. The principle differences between small-scale and large-scale were the large volume of the cells in the cryoprotectant, possibly limiting air exchange, and the longer time required for each step in the process. Both short-cycled and long-cycled cells were tested in a scaled up experiment. Cells were either thawed and pooled from five tubes and plated on 5 plates or 15 tubes were thawed and plated individually. The short-cycled cells recovered within 5 days, with 100% recovery. While taking considerably longer to recover (two weeks), all the plates from the long-ells cycled cells recovered. The possible effect on the cells of being maintained for an extended time in the cryopreservant solutions (due to the time required for dispensing large number of tubes) was also tested. In this experiment, the cells were transferred to tubes and frozen approximately two hours after the first set of samples. There were no obvious differences in the recovery of the cells from the two freezings.

TABLE 1
Cryopreservation of Dicot and Monocot Cell Lines and Transformed
Cell Lines

| Cryopreserved | Genetic | Base | | |
|---|---|---|---|---|
| Line | Event | Cell | Recovery | |
| Number | Number | Line | Status | Comments/Source |
| na | CHN-18 | NT1 | 100% | NDV expressing master seed |
| | | | | optimization lot |
| na | MHN-41 | NT1 | 100% | NDV expressing event |
| na | CHA-13 | NT1 | 100% | AIV expressing event |
| na | CHA-47 | NT1 | 100% | AIV expressing event |
| na | SLT102 | NT1 | 100% | LT-B expressing event |
| D80 | Non- | BY2 | 100% | Seed freeze of non-transformed BY2 |
| | transformed | | | tobacco suspension |
| D81 | Non- | JT-NT1 | 100% | Seed freeze of non-transformed JT- |
| | transformed | | | NT1 tobacco suspension |
| D82 | ncVP2-002 | NT1 | 100% | IBD expressing suspension |
| D83 | 1060[1]-012 | NT1 | 100% | IBD expressing suspension |
| D84 | 1060[1]-020 | NT1 | 100% | IBD expressing suspension |
| D85 | 1060[1]-023 | NT1 | 100% | IBD expressing suspension |
| D86 | 1060[1]-026 | NT1 | 100% | IBD expressing suspension |
| D87 | 1060[1]-028 | NT1 | 100% | IBD expressing suspension |
| D88 | 1060[1]-029 | NT1 | 100% | IBD expressing suspension |
| D89 | 1060[1]-031 | NT1 | 100% | IBD expressing suspension |
| D90 | 1060[1]-033 | NT1 | 100% | IBD expressing suspension |
| D91 | 1060[1]-036 | NT1 | 100% | IBD expressing suspension |
| D92 | 1060[1]-042 | NT1 | 100% | IBD expressing suspension |
| D93 | 1060[1]-043 | NT1 | 100% | IBD expressing suspension |
| D94 | 1060[1]-045 | NT1 | 100% | IBD expressing suspension |
| D95 | 1060[1]-047 | NT1 | 100% | IBD expressing suspension |
| D96 | 1060[1]-048 | NT1 | 100% | IBD expressing suspension |
| D97 | 1060[1]-054 | NT1 | 100% | IBD expressing suspension |
| D98 | 1060[1]-055 | NT1 | 100% | IBD expressing suspension |
| D99 | 1060[1]-057 | NT1 | 100% | IBD expressing suspension |
| D100 | 1060[1]-061 | NT1 | 100% | IBD expressing suspension |
| D101 | 1060[1]-068 | NT1 | 100% | IBD expressing suspension |
| D102 | 1060[1]-067 | NT1 | 100% | IBD expressing suspension |
| D103 | byIBD- | BY2 | 100% | IBD expressing event |
| | 182.C1.S14 | | | |
| D104 | byIBD- | BY2 | 100% | IBD expressing event |
| | 185.C1.S14 | | | |
| D105 | byIBD- | BY2 | 100% | IBD expressing event |
| | 189.C1.S14 | | | |
| D106 | byIBD- | BY2 | 100% | IBD expressing event |
| | 192.C1.S14 | | | |
| D107 | byIBD- | BY2 | 100% | IBD expressing event |
| | 198.C1.S14 | | | |
| D108 | byIBD- | BY2 | 100% | IBD expressing event |
| | 199.C1.S14 | | | |
| D109 | byIBD- | BY2 | 100% | IBD expressing event |
| | 213.C1.S14 | | | |
| D110 | byIBD- | BY2 | 100% | IBD expressing event |
| | 214.C1.S14 | | | |
| D111 | byIBD- | BY2 | 100% | IBD expressing event |
| | 216.C1.S14 | | | |
| D112 | byIBD- | BY2 | 100% | IBD expressing event |
| | 218.C1.S14 | | | |
| D113 | byIBD- | BY2 | 100% | IBD2 expressing event |
| | 224.C1.S14 | | | |
| D114 | byIBD- | BY2 | 100% | IBD expressing event |
| | 226.C1.S14 | | | |
| D115 | byIBD- | BY2 | 100% | IBD expressing event |
| | 231.C1.S14 | | | |
| D116 | byIBD- | BY2 | 100% | IBD expressing event |
| | 233.C1.S14 | | | |
| D117 | byIBD- | BY2 | 100% | IBD expressing event |
| | 234.C1.S14 | | | |
| D118 | byIBD- | BY2 | 100% | IBD expressing event |
| | 237.C1.S14 | | | |
| D119 | byIBD- | BY2 | 100% | IBD expressing event |
| | 238.C1.S14 | | | |
| D120 | byIBD- | BY2 | 100% | IBD expressing event |
| | 239.C1.S14 | | | |

TABLE 2

| | | |
|---|---|---|
| | | Amount |
| Ingredients | Source | per liter |

NT1 Media

| | | | |
|---|---|---|---|
| MS basal salts | Phytotechnology¹ M524 | 100 | ml |
| Myo-inositol | Sigma² I-3011 | 100 | mg |
| Potassium Phosphate Dibasic | Sigma² P-3786 | 137.4 | mg |
| Anhydrous | | | |
| MES | Simga² M-2933 | 0.5 | g |
| 2,4 dichlorophenoxyacetic | Phytotechnology¹ D309 | 222 | μl |
| acid solution (2,4-D) | | | |
| (10 mg/ml) | | | |
| Thiamine/HCL | Sigma² T-3902 | 1 | mg |
| Sucrose | Sigma² S-5309 | 30 | g |
| RO/DI water | | | |

NT1 VP Media

| | | | |
|---|---|---|---|
| MS basal salts | Phytotechnology¹ | 100 | ml |
| | Cat. # M524 | | |
| Modified MS Vitamins | Table 3 | 10 | ml |
| Myo-inositol | Sigma² Cat. # I-3011 | 100 | mg |
| Potassium Phosphate Dibasic | Sigma² Cat. # P-3786 | 137.4 | mg |
| Anhydrous | | | |
| 2-Morpholinoethanesulfonic | Sigma² Cat. # M-2933 | 0.5 | g |
| acid (MES) | | | |
| 2,4-D (10 mg/ml) | Phytotechnology¹ | 222 | μl |
| | Cat. # D309 | | |
| Sucrose | Sigma² Cat. # S-5309 | 30 | g |
| L-Proline (2.5 M) | Sigma² Cat. # P-5607 | 2.4 | ml |
| RO/DI water | | | |

Cryopreservation Media
NT1 VP/CM

| | | | |
|---|---|---|---|
| Sucrose | Sigma² Cat. # S-5309 | 342.27 | g |
| Glycerol | Sigma² Cat. # G-2025 | 46.06 | g |
| DMSO | Sigma² Cat. # I-3011 | 35.5 | ml |
| NT1 VP media | | 226.64 | ml |

¹Phytotechnology Laboratories (Shawnee Mission, KS)
²Sigma-Aldrich (St. Louis, MO)
³Meiji Seika (Kaisha, Japan)

TABLE 3

| Modified MS vitamins (100X) | Per Liter DI water |
|---|---|
| Nicotinic Acid | 5 mg/L |
| Pyridoxin HCL | 50 mg/L |
| Thiamine HCL | 200 mg/L |
| Glycine | 200 mg/L |

TABLE 4
T309 Medium

| | | Catalog | |
|---|---|---|---|
| Ingredient | Source | Number | Amount/L |
| AA Custom Mix | See FIG. 2 | CM024 | 1 package |
| Sucrose | Sigma² | S-5309 | 20.0 gm |
| For selection media add the following | | | |
| Herbiace ™ | Meiji Seika³ | | 600 μl of a 5 mg/ml |
| (bialaphos) | | | soln. |

*Bring to volume and adjust to pH 5.8
¹Phytotechnology Laboratories (Shawnee Mission, KS)
²Sigma-Aldrich (St. Louis, MO)
³Meiji Seika (Kaisha, Japan)

We claim:

1. A method for making a cryopreserved plant cell comprising:
a) passaging cells to about mid-log growth phase for at least 1 passage;
b) concentrating said passaged cells; and
c) adding a cryopreservation media to said concentrated passaged cells to form a cryopreservation composition.

2. The method according to claim 1, further comprising cooling said cryopreservation composition.

3. The method according to claim 1, further comprising freezing said cryopreservation composition.

4. The method according to claim 2, further comprising freezing said cryopreservation composition.

5. The method according to claim 1, wherein the cell is transformed.

6. The method according to claim 1, wherein said cell is selected from the group consisting of cells in Table 1.

7. A method of forming a cryopreservation composition comprising the steps of:
a) growing transformed or non-transformed cells in or on selectable media;
b) inoculating a culture flask containing culture medium with said cells to form a liquid culture and passaging said liquid culture of transformed or non-transformed cells at least 1, 2, 3, 4, 5, 6, 7, 8, 9, or 10 times;
c) recovering said passaged cells; and
d) adding said recovered cells to a cryopreservation media to form a cryopreservation composition.

8. The method according to claim 7, wherein said cells are monocot or dicot plant cells.

9. The method according to claim 8, wherein said cells are transformed.

10. The method according to claim 9, wherein said cells are plant cells.

11. The method according to claim 10, wherein said cells are tobacco cells.

12. The method according to claim 10, wherein said cells are rice cells.

13. The method according to claim 7, wherein said cryopreservation media is formulated in water and comprises 342.27 g of sucrose/L, 46.06 g glycerol/L, 35.5 mL DMSO/L and 226.64 mL of medium chosen from the group selected from NT1 VP medium, VP medium, or T309 medium.

14. A method for cryopreserving transformed plant cells comprising the steps:
a) growing transformed plant cells on callus on selectable media;
b) inoculating a culture flask containing culture medium with said plant cells from said callus to form a liquid culture;
c) culturing said liquid culture to about mid-log growth phase;
d) withdrawing a first volume (VOL1) of said liquid culture grown to about mid-log phase, inoculating said first volume into culture flask containing a second volume (VOL2) of culture medium to form a passage culture and culturing said passage culture to about mid-log growth phase;
e) optionally repeating step d) at least 1, 2, 3, 4, 5, 6, 7, 8, 9, or 10 additional times;
f) recovering plant cells from said passage culture;
g) suspending said plant cells in a volume of a second medium (CULT);
h) adding a volume of cryopreservation media (CRYO) to the suspended plant cells provided in step g) to form a cryopreservation composition;
i) cooling said cryopreservation composition; and
j) freezing said cryopreservation composition.

15. The method according to claim 14, wherein step d) is repeated at least 1, 2, 3, 4, 5, 6, 7, 8, 9, or 10 additional times.

16. The method according to claim 14, wherein said plant cell is tobacco or rice.

17. The method according to claim 15, wherein said plant cell is tobacco or rice.

18. The method according to claim 14, wherein VOL1 and VOL2, independently, range from 1 to at least 20; 1 to at least 10; or 1 to at least 5, inclusive of fractional values between any of these values.

19. The method according to claim 18, wherein VOL 1:VOL2 is 1:3.

20. The method according to claim 18, wherein the ratios of CULT:CRYO are added together and CULT ranges from 1 to 100 and CRYO ranges from 1 to 100.

21. The method according to claim 20, wherein CULT:CRYO is 1:1.

22. The method according to claim 15, wherein the ratios of CULT:CRYO are added together and CULT ranges from 1 to 100 and CRYO ranges from 1 to 100.

23. The method according to claim 22, wherein CULT:CRYO is 1:1

24. The method according to claim 14, further comprising the step of thawing said cryopreservation composition.

25. The method according to claim 24, further comprising the step of recovering and culturing cells from said cryopreservation composition.

26. The method according to claim 18, further comprising the step of thawing said cryopreservation composition.

27. The method according to claim 26, further comprising the step of recovering and culturing cells from said cryopreservation composition.

28. The method according to claim 20, further comprising the step of thawing said cryopreservation composition.

29. The method according to claim 28, further comprising the step of recovering and culturing cells from said cryopreservation composition.

30. The method according to claim 22, further comprising the step of thawing said cryopreservation composition.

31. The method according to claim 30, further comprising the step of recovering and culturing cells from said cryopreservation composition.

32. The method according to claim 14, wherein step d) is repeated 6 times.
